(19) United States
(12) Patent Application Publication
(10) Pub. No.: US 2011/0275709 A1
(43) Pub. Date: Nov. 10, 2011

(54) TELLURIUM-CONTAINING COMPOUNDS FOR AFFECTING MALE'S FERTILITY FOLLOWING CHEMOTHERAPY AND/OR RADIOTHERAPY

(75) Inventors: Benjamin Sredni, Kfar-Saba (IL); Michael Albeck, Ramat-Gan (IL); Benjamin Ron, Savyon (IL)

(73) Assignee: BIOMAS LTD., Kfar-Saba (IL)

(21) Appl. No.: 13/143,546

(22) PCT Filed: Jan. 7, 2010
(86) PCT No.: PCT/IL10/00019

§ 371 Date: Jul. 7, 2011

Related U.S. Application Data
(60) Provisional application No. 61/193,916, filed on Jan. 8, 2009.
(60) Provisional application No. 61/213,654, filed on Jun. 30, 2009.

(51) Int. Cl. A61K 31/33; A61P 15/08; A61P 15/00; C07D 517/22; C07D 329/00

(52) U.S. Cl. 514/462; 549/265; 549/455; 514/467

(57) ABSTRACT

Use of tellurium-containing compounds for maintaining and/or augmenting fertility in a male subject following chemotherapy and/or radiotherapy is disclosed. The tellurium-containing compound is utilized in combination with a chemotherapeutic agent and/or radiation, such that the male subject treated by the chemotherapeutic agent and/or radiation and by the tellurium-containing compound is instructed to refrain from conceptive sex for a pre-determined time period, following the chemotherapy and/or radiotherapy, during which conception is undesired.

### FIELD AND BACKGROUND OF THE INVENTION

The present invention, in some embodiments thereof, relates to a method of maintaining and/or augmenting fertility and, more particularly, but not exclusively, to a method of maintaining and/or augmenting fertility in a male subject undergoing chemotherapy and/or radiotherapy.

Male fertility depends on the proper function of the testes, the male sex organs that produce sperm. Damage to the testes as a result of radiation or chemotherapy is a common cause of sterility among cancer patients. Chemotherapy involves the use of drugs that damage and/or destroy cells that grow rapidly, a characteristic of cancer cells. Unfortunately, chemotherapy also affects normal cells that grow rapidly, such as blood cells in the bone marrow, cells in the hair follicles, and the cells that generate sperm in the testes.

Following cancer chemotherapy most men develop low levels of sperm (oligospermia) or absence of sperm (azoospermia). Most patients undergoing chemotherapy become azoospermic approximately 7-8 weeks after starting treatment [Pont & Albrecht, Fertil Steril 1997; 68:1-5]. In addition, the testicular cells which produce testosterone, Leydig cells, may also be affected by chemotherapy, resulting in low or lack of testosterone production. These conditions may persist for long periods of time and may be permanent.

The mutagenic properties of many forms of chemotherapy are also a concern, as genetic defects appear in sperm cells as a result of such chemotherapy. For example, increased levels of sperm aneuploidy are observed for approximately 100 days following chemotherapy [Robbins et al., Nat-Genet. 1997; 16: 74-78]. In animal studies, chemotherapy-induced aneuploidy and DNA strand breaks have been shown to alter embryonic development [Hales et al., J Natl Cancer Inst Monogr 2005; 34:28-31]. Because of the dangers of genetic defects in sperm, it has been recommended that patients use contraception from the beginning of chemotherapy to 6 months after completion of the treatment [Meistrich, Hum Reprod 1993; 8:8-10; Meseguer et al., Hum Reprod 2003; 18:1281-1285]

The effect of chemotherapy on the testes depends on the type of drug, dose, and schedule of treatment. Chemotherapeutic agents that are toxic to the testes are commonly referred to as gonadotoxic agents. Chemotherapeutic agents (e.g., cyclophosphamide, procarbazine) belonging to the class commonly referred to as “alkylating agents” are particularly toxic to testes. Long-term infertility due to treatment with alkylating agents may be expected in more than 50% of the patients at a cumulative dose of cyclophosphamide greater that 6 grams/m², and procarbazine at a dose greater than 4 grams/m². Procarbazine-containing regimens (MOPP/ABVD) result in azoospermia in the vast majority of patients.

Alkylating agents are commonly used in the treatment of cancer as well as for other diseases, such as autoimmune diseases and in bone marrow ablation.

Tellurium-containing compounds have been shown in both preclinical and clinical studies to have beneficial effects against diverse complications caused by chemotherapeutic agents. Thus, the tellurium-containing compound AS101 was shown to protect mice from hematopoietic damage caused by lethal and sublethal doses of chemotherapeutic drugs, including cyclophosphamide (Cy), and to increase the survival of mice treated with various cytotoxic drugs or radiation, without negatively affecting treatment efficacy [Kalechman et al., Cancer Res 1991; 51:1499-1503; Kalechman et al., Radiat Res 1993; 136:197-204; Kalechman et al., Cancer Res 1993; 53:1838-1844; Kalechman et al., Immunopharmacology 1995; 29:149-158; Sredni et al., Int J Cancer 1996; 65:97-103; Sredni et al., Cancer Res 2004; 64:1843-1852].

Clinical trials in cancer patients treated with AS101 in combination with chemotherapy showed that treatment with AS101 induced a significant reduction in the severity of neutropenia, thrombocytopenia, and alopecia that accompany chemotherapy (Kalechman et al., Exp Hematol 1995; 23:1358-1366; Sredni et al., J Clin Oncol 1995; 13:2432-2353; Sredni et al., Int J Cancer 1996; 65:97-103; Sredni et al., Cancer Res 2004; 64:1843-1852].

In addition, AS101 itself exhibits a clear anti-tumoral effect in a variety of tumor models in mice and humans. AS101 was found to have a synergistic effect with Cy in the treatment of tumor-bearing mice, suggesting that the combination of AS101 and Cy provides a more effective treatment of their tumors [Kalechman et al., Cancer Res 1991; 51:1499-1503].

AS101 sensitizes tumors to chemotherapy by inhibiting the tumor interleukin 10 autocrine loop, which results in decreased Stat3 activity, and by down regulation of the Akt/Survivin pathway [Kalechman et al., Int J Cancer 2000; 86:281-288; Sredni et al., FASEB J 2004; 18:400-402; Hayun et al., Biochem Pharmacol 2006; 72:1423-1434

### SUMMARY OF THE INVENTION

The present inventors have now surprisingly uncovered that tellurium-containing compounds such as AS101 prevent testicular damage caused by chemotherapeutic agents and hence that (i) male patients undergoing chemotherapy should not assume fertility loss during and after chemotherapy, and thus should refrain from unprotected sex; and (ii) male patients can practice reproductive activity (e.g., practice conceptive sex) a relatively short time following chemotherapy.

According to an aspect of some embodiments of the present invention, there is provided a method of maintaining and/or augmenting fertility in a male subject undergoing chemotherapy and/or radiotherapy, the method comprising:

(a) administering to the male subject a therapeutically effective amount of a chemotherapeutic agent and/or radiation;

(b) administering to the male subject a gonadal-protective amount of a tellurium-containing compound;

(c) instructing the male to refrain from conceptive sex for a predetermined time period following administration of the chemotherapeutic agent and/or radiation.

According to some embodiments, the method further comprises:

(d) having the male subject practice reproductive activity (e.g., practice conceptive sex) with a female partner after the end of the predetermined time period.

According to an aspect of some embodiments of the present invention, there is provided a use of a tellurium-containing compound in the manufacture of a medicament for maintaining and/or augmenting fertility in a male subject undergoing chemotherapy and/or radiotherapy, the medicament being for use in combination with a chemotherapeutic agent and/or radiation such that the male subject receiving the chemotherapeutic agent and/or radiation and the tellurium-containing compound is instructed to refrain from conceptive sex for a predetermined time period following administration of the chemotherapeutic agent and/or radiation.

According to some embodiments of the invention, the medicament is further such that after the end of the predetermined time period, the male subject can practice reproductive activity with a female partner.

conceptive sex) a relatively short time following chemotherapy.

According to an aspect of some embodiments of the present invention, there is provided a tellurium-containing compound identified for use in maintaining and/or augmenting fertility in a male subject undergoing chemotherapy and/or radiotherapy, the tellurium-containing compound being for use in combination with a chemotherapeutic agent and/or radiation such that the male subject receiving the chemotherapeutic agent and/or radiation and the tellurium-containing compound is instructed to refrain from conceptive sex for a predetermined time period following administration of the chemotherapeutic agent and/or radiation.

According to some embodiments of the invention, the tellurium-containing compound is further such that after the end of the predetermined time period, the male subject can practice reproductive activity with a female partner.

conceptive sex) a relatively short time following chemotherapy.

According to an aspect of some embodiments of the present invention, there is provided a pharmaceutical composition comprising a tellurium-containing compound and a pharmaceutically acceptable carrier, the composition being identified for use in maintaining and/or augmenting fertility in a male subject undergoing chemotherapy and/or radiotherapy, wherein the tellurium-containing compound is being for use in combination with a chemotherapeutic agent and/or radiation such that the male subject receiving the chemotherapeutic agent and/or radiation and the tellurium-containing compound is instructed to refrain from conceptive sex for a predetermined time period following administration of the chemotherapeutic agent and/or radiation.

According to some embodiments of the invention, the pharmaceutical is being packaged in a packaging material and identified in print, in or on the packaging material, for use in combination with the chemotherapeutic agent and/or radiation, for maintaining and/or augmenting fertility in the male subject undergoing chemotherapy and/or radiotherapy, such that the male subject receiving the chemotherapeutic agent and/or radiation and the tellurium-containing compound is instructed to refrain from conceptive sex for a predetermined time period following administration of the chemotherapeutic agent and/or radiation.

According to some embodiments of the invention, the pharmaceutical further comprises the chemotherapeutic agent.

According to some embodiments of the invention, the pharmaceutical composition is being such that after the end of the predetermined time period, the male subject can practice reproductive activity with a female partner.

According to some embodiments of the invention, the reproductive activity is conceptive sex. According to some embodiments of the invention, the reproductive activity is practiced with an impregnable female.

According to some embodiments of the invention, the reproductive activity is assisted reproduction. According to some embodiments, the female partner is either impregnable female or has fertility problem.

According to some embodiments, the pre-determined time period is less than 6 months.

According to some embodiments, the pre-determined time period is less than 4 months.

According to some embodiments, the pre-determined time period is less than 100 days.

According to some embodiments, the pre-determined time period is less than 3 months.

According to some embodiments, the predetermined time period is less than 2 months.

According to some embodiments, the predetermined time period is less than 1 month.

According to some embodiments, the predetermined time period is such that values of a sperm count, functionality and/or appearance of the male subject at the end of the time period are at least close to normal or reference values.

According to some embodiments, the predetermined time period is such that a sperm DNA structure of the male subject at the end of the time period is at least close to normal.

According to some embodiments, the method further comprises, prior to administering to the male subject the chemotherapeutic agent and/or radiation:

determining values of a sperm count, functionality and/or appearance of the male subject, the values being the reference values.

According to some embodiments, the method further comprises, subsequent to instructing the male subject to refrain from participating is reproduction (e.g., refrain from conceptive sex):

determining values of a sperm count, functionality and/or appearance of the male subject; and

determining if the values of a sperm count, functionality and/or appearance of the male subject are at least close to the reference values.

According to some embodiments of the invention, in the use, composition and tellurium-containing compound described herein, values of a sperm count, functionality and/or appearance of the male subject are determined prior to administering to the male subject the chemotherapeutic agent and/or radiation, the values being the reference values.

According to some embodiments of the invention, values of a sperm count, functionality and/or appearance of the male subject are determined subsequent to instructing the male subject to refrain from conceptive sex, so as to determine if the values of a sperm count, functionality and/or appearance of the male subject are at least close to the reference values.

According to some embodiments, the tellurium-containing compound comprises at least one tellurium dioxo moiety.

According to some embodiments, the tellurium-containing compound has a general formula selected from the group consisting of:

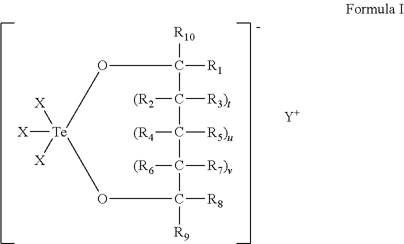

a compound having general Formula II:

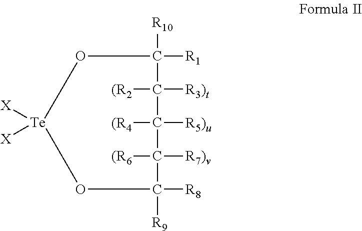

a compound having general Formula III:

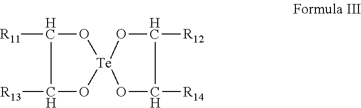

and
a compound having general Formula IV:

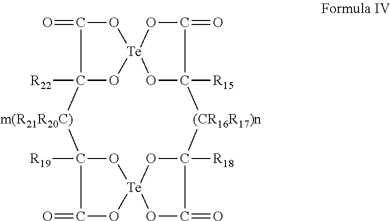

wherein:

each of t, u and v is independently 0 or 1;

each of m and n is independently 0, 1, 2 or 3;

Y is selected from the group consisting of ammonium, phosphonium, potassium, sodium and lithium;

X is a halogen atom; and

each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

According to some embodiments, the tellurium-containing compound has the general Formula I.

According to some embodiments, t, u and v are each 0.

According to some embodiments, each of R₁, R₈, R₉ and R₁₀ is hydrogen.

According to some embodiments, X is chloro.

According to some embodiments, Y is ammonium.

According to some embodiments, the tellurium-containing compound is ammonium trichloro(dioxyethylene-O,O′) tellurate (AS101).

According to some embodiments, the compound has the general Formula IV.

According to some embodiments, each of m and n is 0.

According to some embodiments, each of R₁₅, R₁₈, R₁₉ and R₂₂ is hydrogen.

According to some embodiments, the tellurium-containing compound is SAS.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph presenting the experimental design for testing the effect of AS101 on procarbazine (PCB) induced testicular damage, DNA damage and infertility; animals were administered PBS, AS101, PCB or AS101+PCB, on the days indicated in the graph;

FIGS. 2A-C present photographs of histological sections of mouse testes showing damaged tissue in mice receiving PCB (FIG. 2C) in comparison with tissue of mice receiving either PBS (FIG. 2A) or AS101+PCB (FIG. 2B);

FIG. 3 is a graph showing the relative testicular weight (mg/gram body weight) of mice administered PBS, AS101, PCB or AS101+PCB;

FIG. 4 is a graph showing the litter size of impregnated females mated with male mice administered PBS (control), PCB or AS101+PCB;

FIG. 5 is a graph showing the percentage of females impregnated when mated with male mice administered PBS (control), PCB or AS101+PCB;

FIG. 6 is a graph showing the amount of DNA (1N, 2N or 4N) in testicular cells of mice administered PBS (control), PCB or AS101+PCB;

FIGS. 7A-D present photographs of histological sections of mouse testes showing damaged tissue in mice receiving cyclophosphamide (Cy) (FIG. 7C) in comparison with tissue of mice receiving either PBS (FIG. 7A), AS101 (FIG. 7B) or AS101+PCB (FIG. 7D);

FIG. 8 is a graph showing the percentage of damaged tubules in the testes of mice administered PBS, AS101, Cy or AS101+Cy;

FIG. 9 is a graph showing the percentage of sperm from mice administered PBS, AS101, Cy or AS101+Cy which are characterized by a high DNA fragmentation index (DFI %);

FIG. 10 is a graph showing the percentage of females impregnated when mated with male mice administered PBS, AS101, Cy or AS101+Cy;

FIG. 11 is a graph showing the litter size of impregnated females mated with male mice administered PBS, AS101, Cy or AS101+Cy;

FIGS. 12A-B present a Western blot (FIG. 12B) and quantified results of the Western blot (FIG. 12A) showing levels of phosphorylated Akt (pAkt) in mice administered PBS (control), AS101, Cy or AS101+Cy (α-tubulin was measured as a control); and

FIGS. 13A-B present a Western blot (FIG. 13B) and quantified results of the Western blot (FIG. 13A) showing levels of phosphorylated glycogen synthase kinase-3β (pGSK-3β) in mice administered PBS (control), AS101, Cy or AS101+Cy (α-tubulin and total GSK-3β were measured as controls).

### DESCRIPTION OF SPECIFIC EMBODIMENTS OF THE INVENTION

The present invention, in some embodiments thereof, relates to a method of maintaining and/or augmenting fertility and, more particularly, but not exclusively, to a method of maintaining and/or augmenting fertility in a male subject undergoing chemotherapy and/or radiotherapy.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have surprisingly uncovered that tellurium-containing compounds may substantially reduce and even prevent testicular damage caused by chemotherapeutic agents, thereby minimizing DNA damage in sperm cells and preserving function of the testes. This phenomenon has opened the way for novel and advantageous methods for maintaining and augmenting fertility in males, and reducing a risk of genetic defects in their offspring.

Thus, according to an aspect of some embodiments of the present invention, there is provided a method of maintaining and/or augmenting fertility in a male subject undergoing chemotherapy and/or radiotherapy, the method comprising administering to the male subject a therapeutically effective amount of a chemotherapeutic agent and/or radiation; administering to the male subject a gonadal-protective amount of a tellurium-containing compound; and instructing the male to refrain from conceptive sex for a predetermined time period following administration of said chemotherapeutic agent.

As used herein, the phrase “male subject” refers to an adult male, that is, a male old enough to be biologically capable of fathering offspring.

As used herein, the phrase “maintaining and/or augmenting fertility” describes preventing or reducing the degree of a loss of fertility of the male subject caused by a chemotherapeutic agent. The term “maintaining” herein means preventing a complete loss of fertility, such that at least some fertility remains. The term “augmenting” herein means that a degree of fertility is caused to be higher than would be otherwise (e.g., a partial loss of fertility is prevented or reduced in degree). In some, but not all, embodiments, augmenting fertility comprises restoring a normal level of fertility. The fertility loss may be temporary or permanent. The fertility loss may represent a reduction in the ability to produce offspring and/or a reduction in the likelihood that the offspring of the subject will be healthy (e.g., free from genetic defects). In some embodiments, the loss of fertility is a reduction in the ability to produce offspring which is a result of a choice made as a result of undergoing therapy to refrain from fathering offspring, for example, in order to avoid the risk of fathering offspring with genetic defects.

As used herein, the terms “chemotherapy” and “chemotherapeutic” refer to treatment with a chemical agent capable of causing damage (e.g., cell death and/or DNA mutation) to proliferating cells, typically cancer cells. The chemotherapy may be a treatment for a malignant disease or disorder (e.g., cancer), but chemotherapy for other conditions (e.g., autoimmune diseases, or conditions that require bone marrow ablation) are also intended.

In some embodiments, these terms refer to treatment with chemotherapeutic agents that cause damage to gonadal tissue and/or sperm cells, either as an adverse side effect or per se. Such chemotherapeutic agents are referred to as gonadotoxic agents.

Chemotherapeutic agents suitable for use in embodiments of the present invention, include, without limitation, alkylating agents, vinca alkaloids (e.g., vincristine, vinblastine), antimetabolites (e.g., methotrexate, aminopterin, 5-fluorouracil, cytarabine), topoisomerase interactive agents (e.g., bleomycin, actinomycin, doxorubicin, daunorubicin), paclitaxel and radiotherapeutic agents (e.g., radioactive isotopes).

In some embodiments, the chemotherapeutic agent is an alkylating agent, a vinca alkaloid, an antimetabolite, a topoisomerase interactive agent, or a radiotherapeutic agent, which is a gonadotoxic agent.

According to some embodiments of the present invention, the chemotherapeutic agent is an alkylating agent. Exemplary alkylating agents include, without limitation, nitrogen mustards (e.g., cyclophosphamide, mechlorethamine, uramustine, melphalan, chlorambucil, ifosfamide), nitrosoureas (e.g., carmustine, streptozocin), alkyl sulfonates (e.g., busulfan), thiotepa, platinum-based chemotherapeutic agents (e.g., cisplatin, carboplatin, nedaplatin, oxaliplatin, satraplatin, triplatin), procarbazine, altretamine, dacarbazine, mitozolomide and temozolomide.

In some embodiments, a chemotherapy which would make a male subject infertile comprises administration of an alkylating agent, as described herein.

In some embodiments, the alkylating agent is cyclophosphamide or procarbazine.

As used herein, the terms “radiation” and “radiotherapy” describe any external or internal radiation applied to a tissue to be treated (e.g., for cancer) to which gonadal tissue and/or sperm cells are at least somewhat exposed. In some embodiments, external radiation is applied to the testes or the surrounding area, for example, for treatment of a testicular cancer or prostate cancer. In some embodiments, a radioactive agent (a radiopharmaceutical) is administered systemically to the subject.

As used herein, the phrase “gonadal-protective amount” describes an amount sufficient to result in protection of gonads and/or sperm cells against a damage caused by the chemotherapeutic agent and/or radiation. The protection may be in the form of preventing damage or reducing the degree of damage. In some embodiments, the chemotherapeutic agent and/or radiation decrease an amount of sperm cells, and administration of a gonadal-protective amount of a tellurium-containing compound prevents or at least partially reverses a decrease in the amount of sperm cells. In some embodiments, the chemotherapeutic agent and/or radiation increases an amount of sperm cells having genetic defects, and administration of a gonadal-protective amount of a tellurium-containing compound prevents or at least partially reverses the increase in sperm cells with genetic defects.

As used herein, the phrase “conceptive sex” refers to any form of sexual intercourse (e.g., sexual intercourse without use of contraception) which may result in conception of a child.

The duration of the predetermined time period during which the male subject is instructed, according to embodiments of the present invention, to refrain from conceptive sex will be determined by one of skill in the medical arts based on one or more of relevant factors including, without limitation, the dose, regimen and/or type of chemotherapeutic agent and/or radiation, the risk of genetic defects occurring in any offspring conceived by conceptive sex as a result of the dose, regimen and/or type of the chemotherapeutic agent and/or radiation, the dangers posed by chemotherapeutic agent in seminal fluid to the female sexual partner and/or to a conceived embryo, and the ability of the subject undergoing chemotherapy to withstand exertion.

In some embodiments, the risk of genetic defects occurring in offspring is a factor which at least partially determines the duration of the predetermined time period. It is to be appreciated that in such embodiments, the predetermined time period ends sooner than if a tellurium-containing compound is not administered, because the compound reduces or eliminates damage to DNA of the sperm cells, as exemplified in the Examples section that follows, thereby speeding recovery of normal sperm cells following chemotherapy and/or radiotherapy, or even maintaining normal sperm cells during the entire period following chemotherapy and/or radiotherapy.

Thus, in some embodiments, the predetermined time period is less than 6 months, optionally less than 4 months, optionally less than 100 days, optionally less than 3 months, optionally less than 2 months, and optionally less than 1 month. In some embodiments, the method described herein is utilized for a male subject who would become infertile (e.g., due to a high dose of chemotherapy and/or radiotherapy), and therefore biologically incapable of practicing conceptive sex, as a result of the chemotherapy and/or radiotherapy in the absence of administration of the tellurium-containing compound as described herein. Although infertility appears only after a certain period of time (e.g., about 7 or 8 weeks) has passed from the beginning of the chemotherapy and/or radiotherapy, the subject may be forced to refrain from conceptive sex during the period between the beginning of the chemotherapy and/or radiotherapy and the onset of infertility, due to one or more of the reasons discussed hereinabove for refraining from conceptive sex for a predetermined time period.

Thus, such embodiments of the present invention allow a male subject to practice reproductive activity (e.g., conceptive sex) after the end of the predetermined time period, which would not otherwise be possible for the male subject.

Hence, according to some embodiments, the method is further effected by having said male subject practice reproductive activity with a female partner after the end of the predetermined time period.

As used herein, the term “reproductive activity” refers to an activity that results in generating offspring. Reproductive activity encompasses conceptive sex, as defined herein, or involves assisted reproduction.

In some embodiments, a male can practice conceptive sex with an impregnable female.

The phrase “assisted reproduction” encompasses any reproductive technique that involves artificial or partially artificial means, including those that involve a third party.

Assisted reproduction, as used herein, therefore encompasses any technique by which the process of sexual intercourse is bypassed either by insemination or fertilization of oocytes in the laboratory environment (in vitro fertilization (IVF)).

In vitro fertilization (IVF) is a technique that involves fertilization of the male and female gametes (sperm and egg) which occurs outside the female body.

In vitro fertilisation (IVF) can involve one or more of the following procedures: transvaginal ovum retrieval (OCR); assisted zona hatching (AZH); intracytoplasmic sperm injection (ICSI); autologous endometrial coculture; in zygote intrafallopian transfer (ZIFT); cytoplasmic transfer; and a gestational carrier, as these procedures are described in the art.

Additional assisted reproduction techniques include, but are not limited to, in gamete intrafallopian transfer (GIFT); Artificial insemination (AI); Use of conception devices, such as a conception cap; artificial insemination by donor; surrogacy; reproductive surgery; and in surgical sperm retrieval (SSR).

As discussed hereinabove, the fertility loss in male subjects results from the effect of chemotherapy and/or radiation on various processes associated with fertility. The chemotherapy and/or radiation effect on these processes is reflected by a change in several parameters of a male subject undergoing chemotherapy. These include, for example, sperm count, sperm functionality (e.g., DNA structure of sperm cells) and sperm appearance.

According to some embodiments of the present invention, the predetermined time period is determined such that sperm cells of the male subject will not be considerably damaged at the end of the time period in comparison with normal sperm cells.

In some embodiments, damage to sperm cells is determined by values of a sperm count, sperm functionality and/or sperm appearance, and comparing the sperm count, functionality and/or appearance with that of normal sperm cells, such that the sperm cells are not considered considerably damaged if values of the sperm count, functionality and/or appearance are at least close to normal.

It is to be understood that although the sperm are described herein as being characterized by one or more “values”, qualitative characterization (e.g., whether an appearance of cells appears, to a skilled practitioner, to exhibit damage) as well as quantitative characterization (e.g., numerical values of sperm count, percentage of sperm exhibiting DNA damage) is intended.

Herein, the term “normal” describes the expected characteristics of sperm of the male subject had the subject not undergone chemotherapy or radiotherapy.

In some embodiments, the method as described herein is further effected by determining values of a sperm count, functionality and/or appearance of the male subject prior to administering to the chemotherapeutic agent and/or radiation, and using the obtained values as reference values to define normal sperm cells.

Alternatively, reference values may optionally be obtained by other means, for example, based on average values reported in the medical literature.

As used herein, the phrase “at least close to normal” means±50% of the normal value, optionally ±20%, and optionally ±10%, with respect to numerical values. With respect to qualitative data, the phrase “at least close to normal” optionally means that one of ordinary skill in the art would not recognize that the sperm cells are definitely different, or more damaged, than normal.

Sperm counts, sperm appearance and sperm functionality may be determined by any method commonly used in the art. In some embodiments, the sperm count includes only motile sperm cells (e.g., grade 4 or total of grades 3 and 4 of sperm motility, as these terms are defined by World Health Organization criteria).

Sperm appearance is optionally determined by characterizing a percentage of sperm cells with normal morphology, as defined using World Health Organization criteria.

Sperm functionality is optionally determined by characterizing a DNA structure of sperm (e.g., to assess damage to the DNA structure). Optionally, the DNA structure is analyzed for aneuploidy (e.g., using fluorescent in situ hybridization analysis) and a percentage of aneuploid cells is determined. Alternatively or additionally, a comet assay (e.g., as described by Singh et al. [Exp Cell Res 1988; 175:184-191]) is used to detect damage to the DNA structure. Alternatively or additionally, a sperm chromatin structure assay is used, as described, for example, hereinbelow in the Examples section.

According to some embodiments, the method is further effected by determining values of a sperm count, functionality and/or appearance of the male subject subsequent to instructing said male subject to refrain from conceptive sex, and determining if the values of a sperm count, functionality and/or appearance of said male subject are at least close to the reference values. Optionally, the aforementioned determining of values is performed at or near the end of the predetermined time period in order to confirm that the values have returned to, or are at least close to the reference values, such that the subject may safely practice conceptive sex.

Optionally, if the values at the end of the predetermined time period are not at least close to the reference values, the predetermined time period is followed by a second predetermined time period during which the subject is further instructed to refrain from practicing conceptive sex.

Further according to some embodiments of the invention, there is provided a use of a tellurium-containing compound, as described herein, in the manufacture of a medicament for maintaining and/or augmenting fertility in a male subject undergoing chemotherapy and/or radiotherapy, the medicament being for use in combination with a chemotherapeutic agent and/or radiation such that the male subject receiving such a combined treatment is instructed to refrain from conceptive sex for a predetermined time period following administration of the chemotherapeutic agent and/or radiation, as described herein.

Further according to some embodiments of the invention, there is provided a tellurium-containing compound, as described herein, which is identified for use in maintaining and/or augmenting fertility in a male subject undergoing chemotherapy and/or radiotherapy, in combination with a chemotherapeutic agent and/or radiation, such that the male subject receiving such a combined treatment is instructed to refrain from conceptive sex for a predetermined time period following administration of the chemotherapeutic agent and/or radiation, as described herein.

Further according to some embodiments of the invention, there is provided a pharmaceutical composition which comprises a tellurium-containing compound as described herein and a pharmaceutically acceptable carrier. The pharmaceutical composition is identified for use in maintaining and/or augmenting fertility in a male subject undergoing chemotherapy and/or radiotherapy, in combination with a chemotherapeutic agent and/or radiation, such that the male subject receiving such a combined treatment is instructed to refrain from conceptive sex for a predetermined time period following administration of the chemotherapeutic agent and/or radiation, as described herein.

In some embodiments, the pharmaceutical composition is packaged in a packaging material and identified in print, in or on the packaging material, for use in combination with the chemotherapeutic agent and/or radiation, for maintaining and/or augmenting fertility in a male subject undergoing chemotherapy and/or radiotherapy, such that the male subject receiving the chemotherapeutic agent and/or radiation and the tellurium-containing compound is instructed to refrain from conceptive sex or refrain from sex for a predetermined time period following administration of the chemotherapeutic agent and/or radiation.

In some embodiments of the invention, the pharmaceutical composition further comprises the chemotherapeutic agent, as described herein.

In some embodiments, the tellurium-containing compound and the chemotherapeutic agent are packaged separately in the packaging material.

In some embodiments, the tellurium-containing compound and the chemotherapeutic agent are packaged together, as a single unit dosage form (co-formulation).

The use, tellurium-containing compound and pharmaceutical compositions described herein can further be effected by having the male subject practice reproductive activity after the end of the pre-determined time period, as described herein.

The use, tellurium-containing compound and pharmaceutical compositions described herein can further be such that values of a sperm count, functionality and/or appearance of the male subject are determined prior to administering to the male subject the chemotherapeutic agent and/or radiation, whereby these values are reference values, as described herein. In some embodiments, values of a sperm count, functionality and/or appearance of the male subject are determined subsequent to instructing the male subject to refrain from conceptive sex, so as to determine if the values of a sperm count, functionality and/or appearance of the male subject are at least close to said reference values, as described herein.

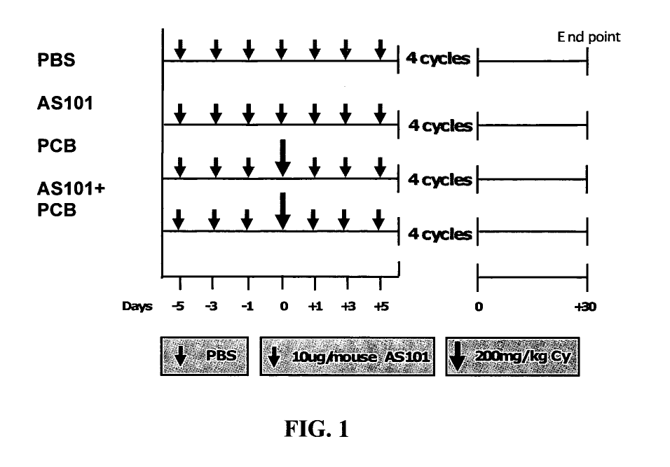

Referring now to the drawings, FIG. 1 describes an exemplary protocol for testing the effect of a tellurium-containing compound (e.g., AS101) on animals being administered a chemotherapeutic agent (e.g., procarbazine).

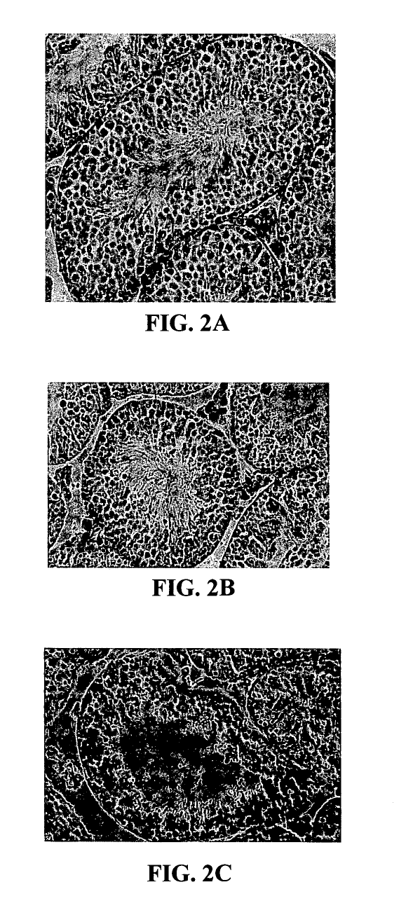

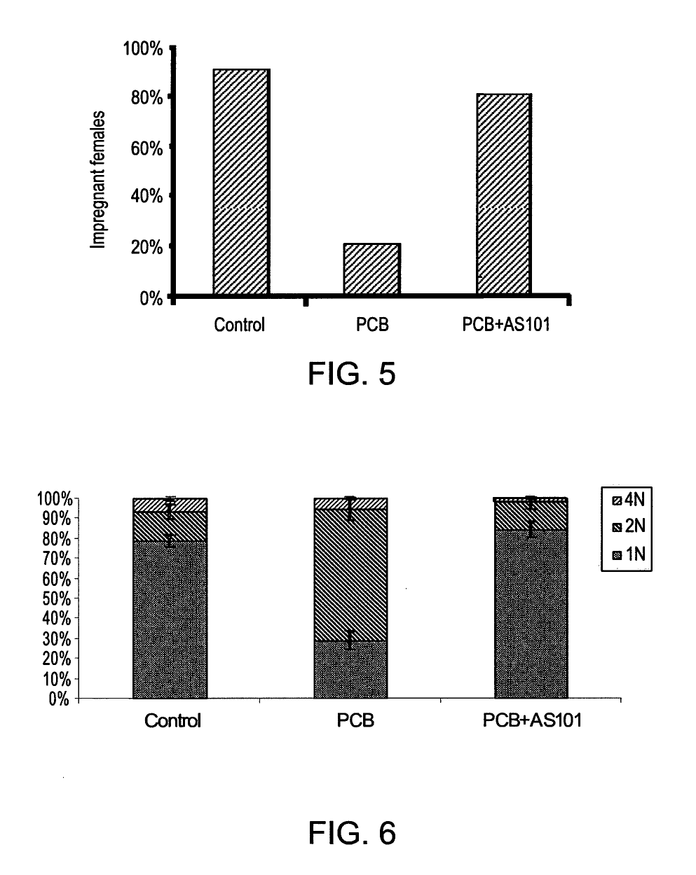

FIGS. 2A-C and 3 show protection by a tellurium-containing compound against procarbazine-induced testicular damage. FIGS. 4 and 5 show protection by a tellurium-containing compound against procarbazine-induced reductions in fertility. FIG. 6 shows protection by a tellurium-containing compound against procarbazine-induced reductions of haploid sperm cells.

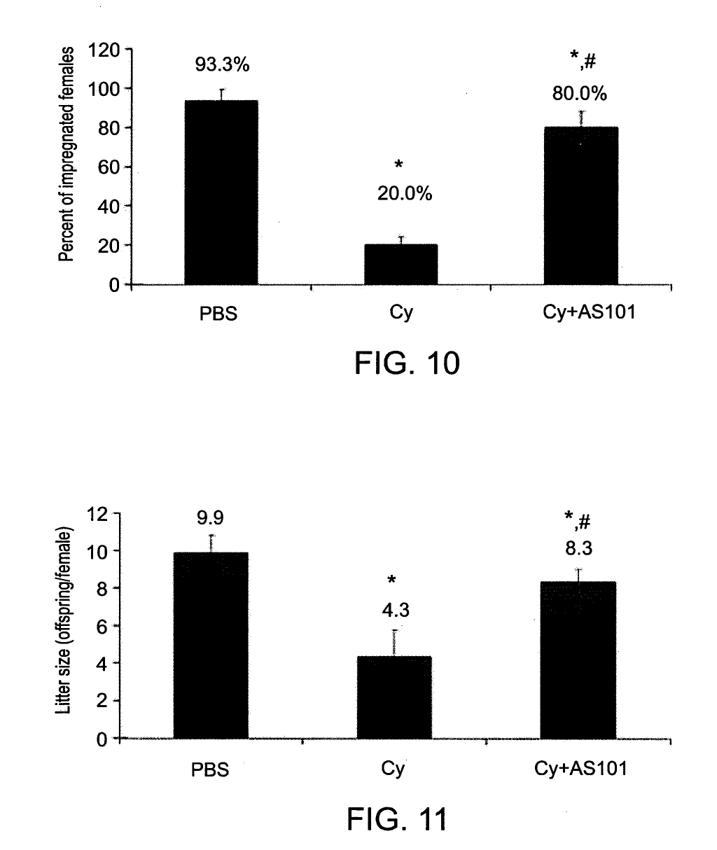

Similarly, FIGS. 7A-D and 8 show protection by a tellurium-containing compound against cyclophosphamide-induced testicular damage. FIG. 9 shows protection by a tellurium-containing compound against cyclophosphamide-induced DNA damage in sperm cells. FIGS. 10 and 11 show protection by a tellurium-containing compound against cyclophosphamide-induced reductions in fertility.

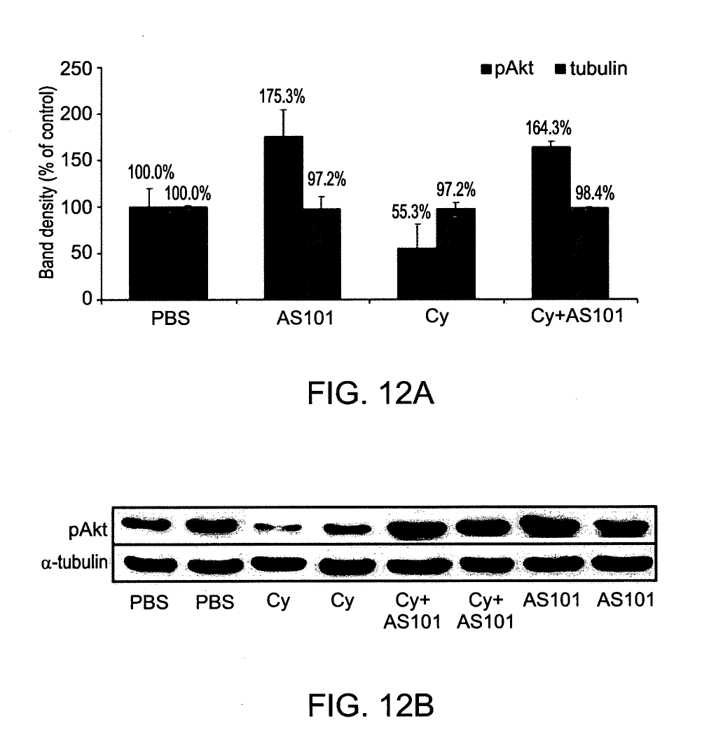

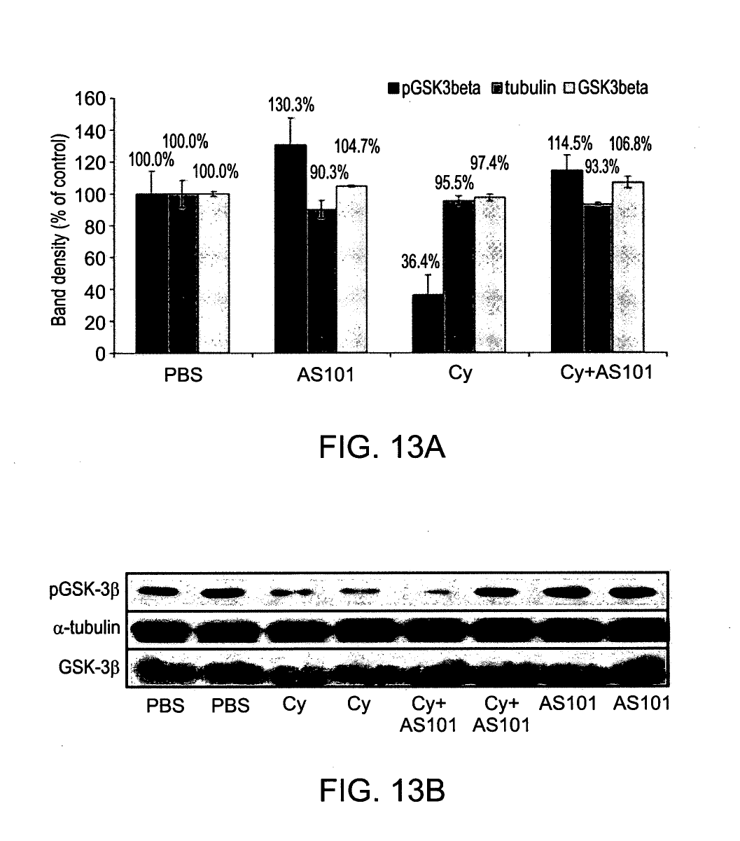

FIGS. 12A-B show that the tellurium-containing compound raises levels of phosphorylated Akt, and FIGS. 13A-B show that the tellurium-containing compound raises levels of phosphorylated GSK-3β.

In any of the methods, compositions and uses described herein, the tellurium-containing compound encompasses any compound that contains one or more tellurium atoms.

In some embodiments, the tellurium-containing compound comprises at least one tellurium dioxo moiety.

Herein throughout, the phrases “tellurium dioxo moiety” and “tellurium dioxide moiety” are used interchangeably, and describe an —O—Te—O—, in which the tellurium center can be further substituted, or a O═Te═O.

The tellurium-containing compound may be an inorganic compound or an organic compound.

Inorganic tellurium-containing compounds include, for example, tellurium dioxide (TeO₂) per se.

Organic tellurium-containing compounds may be in the form of an organic complex such as, for example, a TeO₂ complex with citric acid or ethylene glycol, which may form TeO₂ as an end product in aqueous solutions. A representative example of the latter is the complex TeO₂.HOCH₂CH₂OHNH₄Cl. Otherwise, the tellurium-containing compounds described herein include one or more tellurium atoms and one or more organic moieties that are attached thereto, for example, ammonium salts, or any other salts, of halogenated tellurium-containing compounds having a bidentate cyclic moiety attached to the tellurium atom.

Exemplary compounds in this category can be collectively represented by the general Formula I:

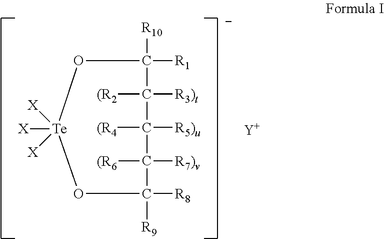

In the general Formula I above, each of t, u and v is independently 0 or 1, such that the compound may include a five-membered ring, a six-membered ring, or a seven-membered ring. In some embodiments, each of t, u and v is 0, such that the compound includes a five-membered ring.

X is a halogen atom, as described hereinabove, and is preferably chloro.

Y can be ammonium, phosphonium, potassium, sodium and lithium, and is preferably ammonium.

Each of R₁-R₁₀ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

As used herein, the term “alkyl” refers to a saturated aliphatic hydrocarbon including straight chain and branched chain groups. In some embodiments, the alkyl group has 1 to 20 carbon atoms. In some embodiments, the alkyl is a medium size alkyl having 1 to 10 carbon atoms. In some embodiments, the alkyl is a lower alkyl having 1 to 5 carbon atoms. The alkyl group may be substituted or unsubstituted. When substituted, the substituent group can be as described herein for R₁.

As used herein, the term “hydroxyalkyl” refers to an alkyl, as this term is defined herein, substituted by a hydroxy group, as defined herein, and includes, for example, hydroxymethyl, hydroxyethyl, hydroxypropyl and hydroxy-n-butyl.

As used herein, the term “halogen”, which is also referred to herein interchangeably as “a halogen atom” or “halo”, includes chloro (Cl), bromo (Br), iodo (I) and fluoro (F).

The term “haloalkyl” refers to an alkyl, as this term is defined herein, substituted by a halogen, as defined herein, and includes, for example, chloromethyl, 2-iodoethyl, 4-bromo-n-butyl, iodoethyl, 4-bromo-n-pentyl and the like.

The term “alkanoyloxy” refers to a carbonyl group, as define herein and includes, for example, acetyl, propionyl, butanoyl and the like.

The term “carboxyalkyl” refers to an alkyl, as this term is defined herein, substituted by a carboxy group, as defined herein, and includes, for example, carboxymethyl, carboxyethyl, ethylenecarboxy and the like.

The term “alkylcarbonylalkyl” refers to an alkyl, as this term is defined herein, substituted by a carbonyl group, as defined herein, and includes, for example, methanoylmethyl, ethanoylethyl and the like.

The term “amidoalkyl” refers to an alkyl, as this term is defined herein, substituted by an amide group, as defined herein, and includes, for example, —CH₂CONH₂; —CH₂CH₂CONH₂; —CH₂CH₂CH₂CONH₂ and the like.

The term “cyanoalkyl” refers to an alkyl, as this term is defined herein, substituted by an cyano group, as defined herein, and includes, for example, —CH₂CN; —CH₂CH₂CN; —CH₂CH₂CH₂CN and the like.

The term “N-monoalkylamidoalkyl” refers to an alkyl, as this term is defined herein, substituted by an amide group, as defined herein, in which one of R′ and R″ is an alkyl, and includes, for example, —CH₂CH₂CONHCH₃, and —CH₂CONHCH₂CH₃.

The term N,N-dialkylamidoalkyl refers to an alkyl, as this term is defined herein, substituted by an amide group, as defined herein, in which both R′ and R″ are alkyl, and includes, for example, —CH₂CON(CH₃)₂; CH₂CH₂CON(CH₂—CH₃)₂ and the like.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be as described herein for R1.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted.

When substituted, the substituent group can be as described herein for R1.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. When substituted, the substituent group can be as described herein for R1.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. Examples, without limitation, include, piperazine, piperidine, morpholine, tetrahydrofuran and tetrahydropyran. The heteroalicyclic may be substituted or unsubstituted. When substituted, the substituent group can be as described herein for R1.

A “hydroxy” group refers to an —OH group.

An “alkoxy” group refers to both an —O-alkyl and an —O-cycloalkyl group, as defined herein.

An “aryloxy” group refers to both an —O-aryl and an —O-heteroaryl group, as defined herein.

A “thiohydroxy” group refers to a —SH group.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “carbonyl” group refers to a —C(═O)—R′ group, where R′ is hydrogen, alkyl, alkenyl, cycloalkyl, aryl, heteroaryl (bonded through a ring carbon) or heteroalicyclic (bonded through a ring carbon) as defined herein.

A “thiocarbonyl” group refers to a —C(═S)—R′ group, where R′ is as defined herein.

A “carboxy” group refers to a —C(═O)—O—R′ or a —O—C(═O)—R′ group, where R′ is as defined herein.

A “sulfinyl” group refers to an —S(═O)—R′ group, where R′ is as defined herein.

A “sulfonyl” group refers to an —S(═O)₂—R′ group, where R′ is as defined herein.

A “sulfate” group refers to a —O—S(═O)₂—OR′ group, where R′ is as defined herein.

A “sulfonamido” group refers to a —S(═O)₂—NR′R″ group or a R′S(═O)₂—NR″, with R′ is as defined herein and R″ is as defined for R′.

A “carbamyl” or “carbamate” group refers to an —OC(═O)—NR′R″ group or a R″OC(═O)—NR′— group, where R′ and R″ are as defined herein.

A “thiocarbamyl” or “thiocarbamate” group refers to an —OC(═S)—NR′R″ group or an R″OC(═S)NR′— group, where R′ and R″ are as defined herein.

An “amino” group refers to an —NR′R″ group where R′ and R″ are as defined herein.

An “amido” group refers to a —C(═O)—NR′R″ group or a R′C(═O)—NR″ group, where R′ and R″ are as defined herein.

A “nitro” group refers to an —NO₂ group.

A “cyano” group refers to a —C≡N group.

The term “phosphonyl” describes a —O—P(═O)(OR′)(OR″) group, with R′ and R″ as defined hereinabove.

The term “phosphinyl” describes a —PR′R″ group, with R′ and R″ as defined hereinabove.

As cited hereinabove, the compounds in this category are salts of organic tellurium-containing compounds. The salts can be, for example, ammonium salts, phosphonium salts and alkaline salts such as potassium salts, sodium salts, lithium salts and the like.

Hence, Y in Formula I above can be a phosphonium group, as defined herein, an ammonium group, as defined herein, potassium (K⁺), sodium (Na⁺) or lithium (Li⁺).

As used herein, the term “phosphonium” describes a —P⁺R′R″R′″ group, with R′ and R″ as defined herein and R′″ is as defined for R′. The term “phosphonium”, as used herein, further refers to a —P⁺R₆ group, wherein each of the six R substituents is independently as defined herein for R, R″ and R′″.

The term “ammonium” describes a —N⁺R′R″R′″ group, with R′, R″ and R′″ as defined herein.

In some embodiments, compounds in this category include compounds having the general Formula I described above, in which Y is ammonium or phosphonium, t, u and v are each 0, and each of R₁, R₈, R₉ and R₁₀ is independently hydrogen or alkyl. These compounds can be represented by the following structure:

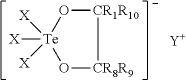

wherein each of R₁, R₈, R₉ and R₁₀ is independently hydrogen or alkyl, whereas, in some embodiment, the alkyl is methyl, and X is halogen, preferably chloro.

In some embodiments, a tellurium-containing compound for use in the context of the present embodiments has the following structure:

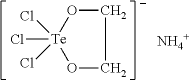

This compound is ammonium trichloro(dioxyethylene-O,O′)tellurate, which is also referred to herein and in the art as AS101.

Additional representative examples of organic tellurium-containing compound that are suitable for use in the context of the present invention include halogenated tellurium having a bidentate cyclic moiety attached to the tellurium atom. The bidentate cyclic moiety is preferably a dioxo ligand having two oxygen atoms attached to the tellurium atom.

Exemplary compounds in this category can be represented by the general Formula II:

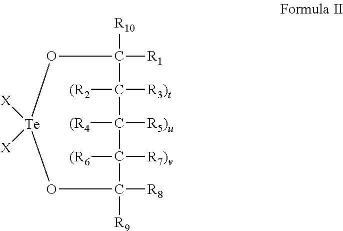

wherein t, u, v, X and R₁-R₁₀ are as defined hereinabove.

In some embodiments, the tellurium-containing compounds are those in which t, u, and v are each 0, and X is chloro, such as, but not limited to, the compound having the following structure:

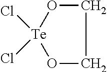

The above compound is also known in the art and referred to herein as AS103.

The organic tellurium-containing compounds having Formulae I and II can be readily prepared by reacting tetrahalotelluride such as TeCl₄ with a dihydroxy compound, as is described in detail in U.S. Pat. Nos. 4,752,614, 4,761,490, 4,764,461 and 4,929,739, which are incorporated by reference as if fully set forth herein.

Additional representative examples of organic tellurium-containing compounds that are suitable for use in the context of the present embodiments include compounds in which two bidentatic cyclic moieties are attached to the tellurium atom. Preferably, each of the cyclic moieties is a dioxo moiety.

Exemplary compounds in this category are collectively represented by the general Formula III:

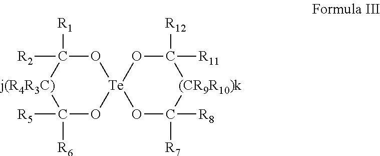

In the general Formula III above, each of j and k is independently an integer from 0 to 4, such that the compound may include a five-membered ring, a six-membered ring, a seven-membered ring, an eight-membered ring and/or a nine-membered ring. In some embodiments, each of j and k is an integer from 0 to 2, such that the compound includes a five-membered ring, a six-membered ring and/or a seven-membered ring. In some embodiments, each of j and k is 0.

R₁-R₁₂ are as defined hereinabove for R₁-R₁₀.

In some embodiments, tellurium-containing compounds in this category are those in which j and k are each 0, and R₃, R₄, R₉ and R₁₀ are each hydrogen, having the following structure:

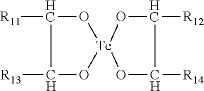

wherein each of R₁₁-R₁₄ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido, as these terms are defined herein.

In some embodiments, a tellurium-containing compound in this category is a compound in which each of R₁₁-R₁₄ is hydrogen. This compound is also known in the art and referred to herein as AS102.

Additional representative examples of organic tellurium-containing compounds that are suitable for use in the context of the present embodiments include the recently disclosed ditellurium compounds having general Formula IV:

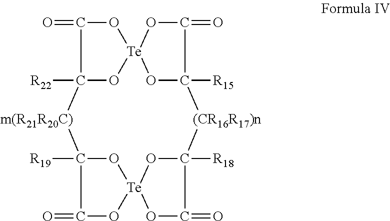

wherein each of R₁₅-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, alkoxy, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido, as these terms are defined herein; and

m and n are each an integer from 0 to 3.

Exemplary compounds in this category are those in which m and n are each 0.

An exemplary compound in this family is a compound in which R₁₅, R₁₈, R₁₉ and R₂₂ are all hydrogen, referred to hereinafter as SAS, and which has the following structure:

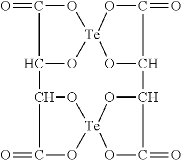

According to some embodiments of the present invention, the tellurium-containing compound is either AS101 or SAS, as described herein.

The compounds described above can be administered or otherwise utilized in the various aspects of the present invention, either as is or as a pharmaceutically acceptable salt thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

The tellurium-containing compound and the chemotherapeutic agent (and/or radiation), utilized in embodiments of the methods and uses described herein, may be administered concomitantly. Alternatively, the tellurium-containing compound may be administered before or after the chemotherapeutic agent and/or radiation (i.e., sequentially).

In any of the methods and uses described herein, administration of the tellurium-containing compound and optionally of additional active agents (e.g., the chemotherapeutic agent) can be performed via various routes of administrations.

Suitable routes of administration may, for example, include the inhalation, oral, buccal, rectal, transmucosal, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a tissue region of a subject.

Determination of a gonadal-protective amount of a tellurium-containing compound is well within the capability of those skilled in the art.

For any preparation used in the methods of the invention, the gonadal-protective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity of the potential damage caused by the chemotherapy and/or radiotherapy, dosing can be of a single or a plurality of administrations.

When administering systemically, a therapeutically effective amount of the tellurium-containing compounds described herein may range, for example, from about 0.01 mg/m²/day to about 20 mg/m²/day and thus can be for example, 0.01 mg/m²/day, mg/m²/day, 1 mg/m²/day, 2 mg/m²/day, 3 mg/m²/day, 4 mg/m²/day, 5 mg/m²/day, and up to 10 mg/m²/day. Preferably, for systemic administration, a gonadal-protective amount of a compound of formula I, II, III or IV ranges from about 0.01 mg/m²/day to about 10 mg/m²/day. Higher gonadal-protective amounts, such as, for example, up to 20 mg/m²/day can also be employed.

In one embodiment, when administered intraperitoneally, the gonadal-protective amount is 0.01 mg/m²/day and higher and thus can be, for example, 0.01 mg/m²/day, 0.05 mg/m²/day, 0.1 mg/m²/day, 0.2 mg/m²/day, 0.5 mg/m²/day, 0.6 mg/m²/day, 0.7 mg/m²/day, 0.8 mg/m²/day, 0.9 mg/m²/day, 1 mg/m²/day, 2 mg/m²/day, 3 mg/m²/day, 4 mg/m²/day, 5 mg/m²/day, and up to 20.0 mg/m²/day. When administered orally in humans, a daily dose typically ranges between 0.1 mg and 200 mg, more preferably between 1 mg and 100 mg, depending on the age and weight of the subject. The total daily dose may be administered as a single dosage, or may be divided into a number of separate doses.

In any of the methods and uses described herein, the tellurium-containing compound and the chemotherapeutic agent can form a part of a pharmaceutical composition (either each alone or in combination), which further comprises a pharmaceutically acceptable carrier.

Pharmaceutical compositions comprising one or more tellurium-containing compound described herein may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with embodiments of the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients may be formulated in aqueous solutions, preferably in physiologically compatible buffers such as Hank's solution, Ringer's solution, or physiological salt buffer.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise glass, plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

In one embodiment, the pharmaceutical composition described herein is packaged in a packaging material and identified in print, in or on said packaging material, for use in the maintenance and/or augmentation of fertility of a male subject undergoing chemotherapy and/or radiotherapy, as described herein. Optionally, the pharmaceutical composition is further identified for use in combination with the chemotherapeutic agent used for chemotherapy and/or the radiation.

In one embodiment, a concentration of tellurium-containing compound in the carrier ranges from about 0.01 weight percent to about 50 weight percents, more preferably from about 0.1 weight percent to about 25 weight percents, of the total weight of the composition.

It is expected that during the life of a patent maturing from this application many relevant chemotherapeutic agents will be developed and the scope of the term “chemotherapeutic agent” is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Methods

Materials:

Procarbazine (PCB) and cyclophosphamide (Cy) were obtained from Sigma, and were dissolved in phosphate buffered saline (PBS) to a concentration of 25 mg/ml.

AS101 was prepared as described in U.S. Pat. Nos. 4,752,614, 4,761,490, 4,764,461 and 4,929,739, dissolved in PBS to a concentration of 50 μg/ml, and stored at 4° C.

RIPA lysis buffer, protease inhibitor cocktail and phosphatase inhibitor cocktail II were obtained from Sigma.

Rabbit anti-human phospho-Akt1/2/3 (Ser473, sc-7985-R) monoclonal antibodies were obtained from Santa Cruz (Santa Cruz, Calif., USA) and diluted 1:400 in Tris buffered saline with 0.1% TWEEN and 5% bovine serum albumin.

Rabbit anti-human GSK-3β (Ser9, #9315) and anti-human phospho-GSK-3β (Ser9, #9336) monoclonal antibodies were obtained from Cell Signaling (Danvers, Mass., USA) and diluted 1:300.

Mouse anti-α-tubulin monoclonal antibody (T6199) was obtained from Sigma.

Peroxidase-conjugated goat anti-rabbit IgG (#111-035-003) and anti-mouse IgG (#115-035-003) antibodies were obtained from Jackson Laboratories (Bar Harbor, Me., USA) and diluted 1:5,000.

Antibodies against phospho-proteins were diluted in Tris buffered saline with 0.1% Teen and 5% bovine serum albumin, whereas all other antibodies were diluted in Tris buffered saline with 0.1% TWEEN and 1% skim milk.

Falcon tubes were obtained from BD Immunocytometry Systems (San Jose, Calif., USA).

Animals:

Inbred Balb/c male mice, aged 5-6 weeks, were used. To ensure the fertility of the mice, each mouse was placed with two young healthy females. Only males that succeeded in fertilizing at least one female, thereby demonstrating fertility, were chosen for the experiment.

Mice were housed in the animal center at the SPF animal housing facility at Bar Ilan University. Ethical approval of animal experimentation was received from the Institutional Ethics Committee.

Evaluation of Male Fertility:

Fertility assays were performed on the “chemotherapy only” group, “chemotherapy+AS101” group, and control (PBS) group. Each male was placed with three young virgin females, aged 6 weeks. These females were examined daily for vaginal plugs. Once mating was documented, females were separated, and the pregnancies allowed to progress. Females that did not show evidence of mating were separated at the end a 30-day period and sacrificed. The number of pregnant females and litter size was noted.

Sperm Extraction:

In order to recover the motile epididymal spermatozoa, the two caudal epididymides were placed in M2 medium (Sigma), minced with fine scissors and incubated at 37° C. in an atmosphere of 95% air and 5% CO₂ for 15 minutes prior to careful removal of all medium. Samples were then diluted to a final volume of 1 ml in M2. The concentration of sperm cells in the medium was then evaluated using a hemocytometer, and the samples were frozen at −80° C.

Flow Cytometry Sperm Chromatin Structure Assay (SCSA):

To measure DNA damage to sperm, the susceptibility of sperm nuclear DNA to low pH-induced denaturation in situ was assayed as described by Evenson et al. [J. Androl. 2002; 23:25-43]. Briefly, frozen sperm samples were thawed and diluted to a concentration of 1-2×10⁶ sperm/ml with TNE buffer solution (0.15 M NaCl, 0.01M Tris-HCl and 1 mM disodium EDTA (ethylenediaminetetraacetic acid), pH 7.4). From the diluted samples, 200 μl was then placed in a Falcon tube and mixed with 400 μl low pH detergent solution (0.1% Triton X-100, 0.15 M NaCl and 0.08 N HCl, pH 1.2). After 30 seconds, the cells were stained with 1.2 ml of acridine orange (AO) staining solution containing 6 μg/ml of AO in staining buffer (0.1M citric acid, 0.2 M Na₂HPO₄, 1 mM EDTA and 0.15 M NaCl, pH 6.0). Samples were analyzed using a FACScan flow cytometry device (BD Immunocytometry Systems). A total of 1×10⁴ spermatozoa from each sample were collected and analyzed at a rate of 100-200 cells per second. Green fluorescence emission of AO was measured at 515-530 nm with a band pass filter and red fluorescence of AO was detected through a 630-650 nm long-pass filter. The resulting fluorescence was quantified by FlowJo software (TreeStar, Oreg., USA). The extent of DNA denaturation of each cell was quantified by the calculated parameter DNA fragmentation index (DFI) using the following formula: red fluorescence/(red+green fluorescence). The percentage of cells with abnormal chromatin structure was defined as the percentage of cells with high DFI values (% DFI).

Analysis of Testicular Damage by DNA Flow Cytometry:

Quantification of DNA content in testicular cell populations by propidium iodide staining followed by flow cytometry is an objective, rapid, and robust method for the analysis spermatogenic damage. The method provides quantitative values for evaluating the changes in the proportion in the number of 1N (mature and immature haploid), 2N (diploid), and 4N (tetraploid) cells in the testis. The method was performed as previously described (Malkov at al., 1998). Briefly, testes were dissected, decapsulated, and the contents of the testes were transferred into a tube containing 5 ml ice-cold separation medium. Then, a collagenase solution was added and incubation was carried out for 5 minutes at 37° C. under vigorous shaking. The seminiferous cords were allowed to sediment to the bottom of the tube while being incubated on ice. The seminiferous cords were washed twice in 10 ml separation medium, re-suspended in separation medium containing 2.5 mg/ml trypsin and 1U/ml DNase I, incubated for 2 minutes at 37° C., and transferred to ice. Using a Pasteur pipette, the seminiferous cords was disintegrated into single cells and were then filtered through a 50-mm nylon mesh, washed twice with separation medium (centrifugation at 200-300 g), and counted. For FACS analysis, testicular cells were brought to a concentration of 2-3×10⁶ cells/ml in separation medium and diluted 1:1 with propidium iodide solution (10 mM Tris pH 8, 1 mM NaCl, 0.1% Nonidet P-40, 0.7 mg/ml Rnase A, and 0.05 mg/ml propidium iodide). Cells were analyzed by a Becton-Dickinson (San Francisco, Calif.) FACSort instrument, equipped with an argon laser, within 2 hours from staining. Excitation was at 488 nm and emission at 585 nm. The parameters measured for each cell were forward scatter (FSC-H), side scatter (SSC-H), and total fluorescence emitted from the cell (FL2-A).

Histological Evaluation of Testicular Damage:

The removed testes were pierced with a needle and fixed in Bouin's fixative. After 24 hours, testes were washed three times and maintained in 70% ethanol. Samples were then embedded in paraffin and sectioned. Tissue sections (5 μm) were then stained using hematoxylin/eosin and examined randomly under a light microscope (AX70, Olympus, Tokyo, Japan) under ×100 magnification.

Gel Electrophoresis and Western Blotting:

Frozen testes were homogenized in 1 ml of RIPA lysis buffer containing protease inhibitor cocktail and phosphatase inhibitor cocktail II. The tissue was then pulverized with a Dounce homogenizer and the homogenate subjected to centrifugation at 14,000 g for 15 minutes. The supernatant was collected and used for protein determination using Bradford reagent. Tissue samples were denatured in reducing buffer (62 mM Tris [pH 6.8], 10% glycerol, 2% sodium dodecyl sulfate [SDS], 5% f3-mercaptoethanol and 0.003% bromophenol blue) and separated by electrophoresis on an SDS (12%) polyacrylamide gel at 35 mA. The separated proteins were transferred onto a nitrocellulose membrane using the transfer buffer (39 mM glycine, 48 mM Tris [pH 8.3] and 20% methanol) at 20 V at 4° C. overnight. The membranes were stained with Ponceau S (0.005% in 1% acetic acid) to confirm equal amounts of protein, and blocked with 5% non-fat dry milk in Tris buffered saline with 0.1% TWEEN (TBS-T) for 1 hour at room temperature, and washed three times for 10 minutes each time in TBS-T. The following primary rabbit monoclonal antibodies were used: anti-human phospho-Akt1/2/3, anti-human GSK-3β and anti-human phospho-GSK-3β. Mouse antibody against chicken α-tubulin was used in order to confirm that the protein load was similar in all lanes. Membranes were incubated with the appropriate diluted antibody overnight at 4° C. After being washed three times for 10 minutes each time in TBS-T, the membranes were incubated for 1 hour at room temperature with peroxidase-conjugated goat anti-rabbit or goat anti-mouse IgG antibody. After washing, the membranes were analyzed by an enhanced chemiluminescence system according to the manufacturer's protocol (Pierce). Densitometry was conducted in order to quantify differences in band intensity using the Image J image-processing program (NIH, Bethesda, Md., USA). Results were normalized by comparison to the values in the PBS control group in each membrane.

Statistical Analysis:

Student's t-test with Bonferroni correction was performed to assess differences between groups. In order to adjust for the dependent study design of the mating experiments, a generalized logistic model, which takes the analysis of the cluster structure of the experiment into consideration, was performed according to Rao and Scott [Biometrics 1992; 48:577-5851. Differences in litter sizes were assessed with the Kruskal-Wallis ANOVA test. Statistical significance was determined at P<0.05.

### Example 1

### Protective Effect of AS101 Against Procarbazine-induced Testicular Damage, DNA Damage and Infertility

The mice in one group (PCB group) were injected intraperitoneally (ip) with 200 mg/kg of procarbazine (PCB) once a week for a period of 5 weeks. The mice in another group (AS101+PCB group) were co-treated with PCB as described for the PCB group and with ip injections of AS101 at a dosage of 10 μg/mouse every other day, starting 1 week before the first PCB injection and continuing during all the weeks of PCB treatment. Other mice were injected with PBS only (PBS group), or only with ip injections of AS101 at a dosage of 10 μg/mouse every other day (AS101 group). The animals from all groups were sacrificed 5 weeks after the last injection by cervical dislocation, and body weights were determined; thereafter, epididymides and testes were quickly dissected out, weighed and fixed. The administration protocols for the four groups are depicted in FIG. 1.

Results of Histological Evaluation of Testicular Damage:

The effect of AS101 co-treatment on the testicular damage induced by three weekly injections of 200 mg/kg PCB was evaluated by histological examination, as described in the Materials and Methods section. Representative histological sections of testes from the PBS group, AS101+PCB group and PCB group are shown in FIGS. 2A, 2B and 2C, respectively. Each group contained 5 mice.

As shown in FIGS. 2A-2C, the testes of mice treated with AS101 and PCB (FIG. 2B) resembled those of mice treated with PBS (FIG. 2A), whereas the testes of mice treated with PCB (FIG. 2C) were characterized by empty and atrophic seminiferous tubules.

Effect of Treatments on Testicular Weight:

PCB was administered to mice in a single dose of 200 mg/kg, and the testes of mice from each of the four groups described hereinabove (PBS, AS101, PCB and AS101+PCB) were weighed to evaluate testicular damage. Weights were normalized to the weight of the mouse. Each group contained 10 mice.

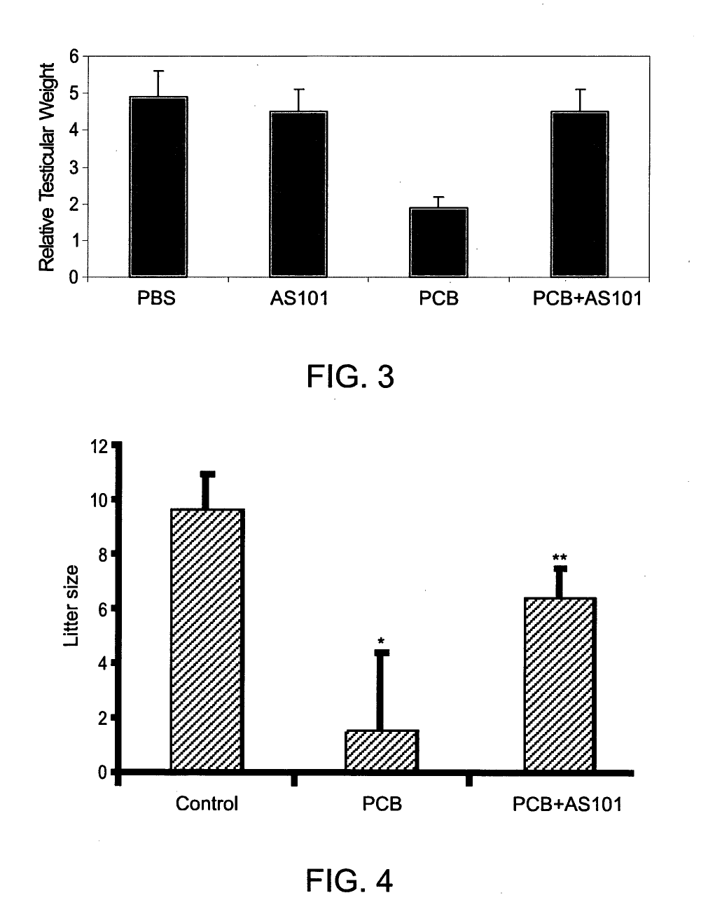

As shown in FIG. 3, PCB caused the average testicular weight to decrease considerably to 1.9±0.2 mg per grams body weight, as compared to 4.9±0.2 mg per grams body weight in PBS-treated mice. In contrast, co-treatment with AS101 significantly protected against the PCB-induced reduction in testicular weight, resulting in an average testicular weight of 4.5±0.4 mg per grams body weight. As101 alone had no significant effect. The average testicular weight of PCB-treated mice was significantly different (P<0.05) than that of both PBS-treated mice and AS101+PCB-treated mice.

Results of Evaluation of Male Fertility:

Male fertility of mice in PBS, PCB and AS101+PCB groups was evaluated as described in the Materials and Methods section. PCB was administered in a single dose of 200 mg/kg. Each group contained 5 male mice.

As shown in FIG. 4, PCB reduced litter size considerably, from 9.5±1.4 in control mice to 1.4±2.9 (P<0.05), while co-treatment of AS101 with PCB resulted in considerably larger litter sizes (6.3±1.6) than did treatment with PCB alone (P<0.05).

As shown in FIG. 5, PCB reduced the percentage of impregnated females considerably, from 90% to 20%, while co-treatment of AS101 almost completely negated the PCB-induced prevention of impregnation, with the percentage of impregnated females in the AS101+PCB group being 80%.

Results of Analysis of Testicular Damage by DNA Flow Cytometry:

Testicular damage of mice in PBS, PCB and AS101+PCB groups was evaluated by DNA flow cytometry as described in the Materials and Methods section. PCB was administered in a single dose of 200 mg/kg. Each group contained 5 mice.

As shown in FIG. 6, the percentage of 1N (haploid) cells in PCB-treated mice was significantly lower (P<0.05) than that of control mice, while the percentage of 2N (diploid) cells was significantly higher (P<0.05), whereas mice treated with AS101 and PCB had similar levels of 1N cells as those of control mice, and significantly more (P<0.05) 1N cells than mice treated with PCB alone.

The distribution of cells with 1N (haploid), 2N (diploid), and 4N (tetraploid) amounts of DNA in the testes of the control animals was 69.7±2.7% 1N, 12.9±4.0% 2N, and 6.1±3.5% 4N. In the PCB-treated animals, the percentage of cells with 1N dropped to 24.2±4.0%, the percentage of 2N cells was 54.9±4.2%, and the percentage of 4N cells was 4.7±0.4%. In the AS101+PCB group, the percentage of cells with 1N was 73.8±3.5%, the percentage of cells with 2N was 12.0±3.2%, and the percentage of cells with 4N was 1.8±0.8%.

The above results indicate that AS101 is effective in protecting against procarbazine-induced testicular damage, DNA damage and infertility.

### Example 2

### Protective Effect of ASl01 against Cyclophosphamide-induced Testicular Damage, DNA Damage and Infertility

Ten mice were injected intraperitoneally (ip) with 200 mg/kg of cyclophosphamide (Cy) once a week for a period of 5 weeks (Cy group). This dose has previously been shown to have a devastating effect on male fertility and corresponds to a therapeutic dose in humans [Elangovan et al., Toxicology 2006; 222:60-70].

Another ten mice (Cy+AS101 group) were co-treated with Cy as described for the Cy group and with i.p. injections of AS101 at a dosage of 10 μg/mouse every other day, starting 1 week before the first Cy injection and continuing during the 5 weeks of Cy treatment.

An additional group of ten mice were injected with PBS only (PBS group), and another ten mice were treated with i.p. injections of AS101 at a dosage of 10 μg/mouse every other day (AS101 group), as described for the Cy+AS101 group. The animals from all groups were sacrificed 5 weeks after the last injection by cervical dislocation, and body weights were determined; thereafter, epididymides and testes were quickly dissected out, weighed and fixed.

Effect of Treatments on Reproductive Organ Weight and Sperm Count:

Body weight, testes weight, testes weight normalized to body weight, epididymis weight, epididymis weight normalized to body weight, and sperm concentration were determined for the mice of each of the 4 treatment groups.

As shown in Table 1, Cy treatment caused a statistically significant decrease in each of the aforementioned parameters as compared to the PBS and AS101 control groups, whereas AS101 co-administered with Cy resulted in a statistically significant increase in each parameter as compared to treatment with Cy alone.

Despite the Cy-induced weight loss, no deaths occurred.

TABLE 1

| | PBS | | | Cy + |
|---|---|---|---|---|
| | (control) | AS101 | Cy | AS101 |
| Body weight | 26.0 ± 0.6 | 26.2 ± 1.0 | 22.8 ± 2.1* | 25.6 ± 1.1# |
| (gram) | | | | |
| Testes | 107.2 ± 12.7 | 105.7 ± 14.7 | 46.3 ± 8.7* | 73.5 ± 7.1*# |
| weight (mg) | | | | |
| Testes | 4.15 ± 0.5 | 4.3 ± 0.3 | 1.98 ± 0.16* | 2.9 ± 0.3*# |
| weight | | | | |
| (mg/gram | | | | |
| body weight) | | | | |
| Epididymis | 39.1 ± 4.8 | 34.2 ± 4.2 | 25.1 ± 2.9* | 31.5 ± 2.0# |
| weight (mg) | | | | |
| Epididymis | 1.5 ± 0.2 | 1.3 ± 0.1 | 1.1 ± 0.1* | 1.2 ± 0.1*# |
| weight | | | | |
| (mg/gram | | | | |
| body weight) | | | | |
| Sperm | 22.1 ± 5.4 | 22.5 ± 3.8 | 8.7 ± 1.6* | 15.6 ± 2.9*# |
| concen- | | | | |
| tration | | | | |
| (10⁶ | | | | |
| cells/ml) | | | | |

*P < 0.05 compared with PBS control group;
#P < 0.05 compared with Cy group

Results of Histological Evaluation of Testicular Damage:

Representative cross sections are shown in FIG. 7A-7D.

As shown in FIG. 7C, the cross-sections of Cy-treated mice were characterized by empty and atrophic seminiferous tubules, compared with the normal cellular content in samples obtained from PBS and AS101-treated mice (FIGS. 7A and 7B, respectively).

As shown in FIG. 7D, damage to seminiferous tubules was considerably less severe in the Cy+AS101 group, with many tubules showing undamaged spermatogenesis.

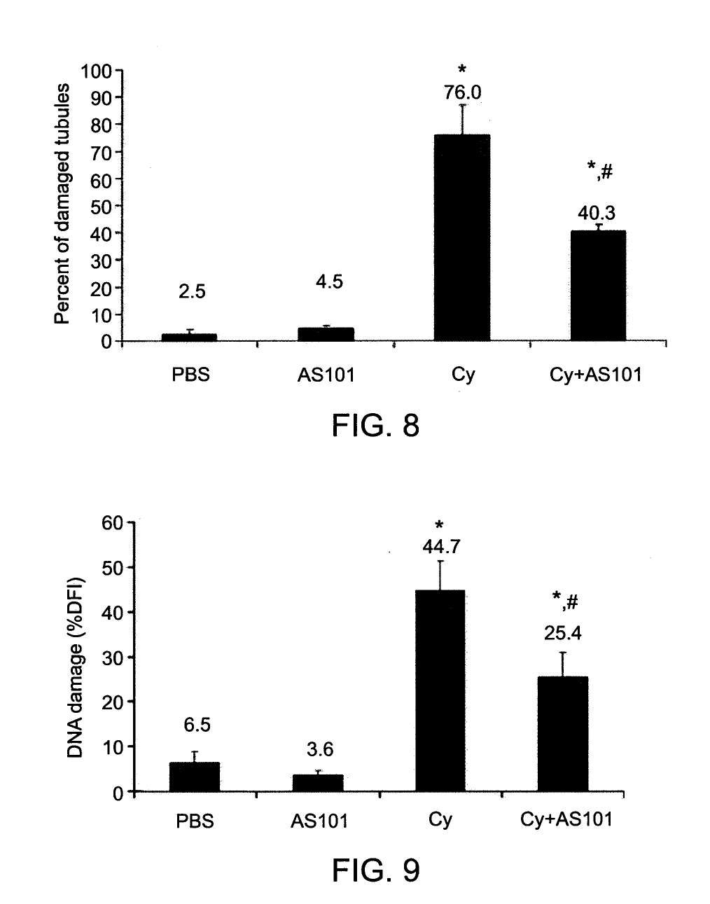

As shown in FIG. 8, the average percentage of damaged seminiferous tubules in the Cy group (76.0±10.8%) was significantly higher than the average percentage of damaged seminiferous tubules in the PBS group (2.5±1.7%), while samples from Cy+AS101-treated animals showed significantly fewer damaged tubules (40.3±2.6%) than in the Cy group.

As shown in FIG. 7B and in FIG. 8, administration of AS101 alone had no effect on testicular histology.

Results of Sperm Chromatin Structure Assay:

Sperm cells were assayed by flow cytometry for the presence of abnormal chromatin structure, as described in the Materials and Methods section. Sperm chromatin damage has been shown to reduce fertilization rates and cause post-implantation embryo loss in animals [Codrington et al., Hum Reprod 2007; 22:1431-1442; Elangovan et al., Toxicology 2006; 222:60-70]

As shown in FIG. 9, administration of Cy caused a significant increase in the percentage of sperm cells with abnormal chromatin structure (% DFI), from 6.5±2.3% in the PBS control group to 44.7±1.0% in the Cy group, whereas in the Cy+AS101 group, the % DFI value was 25.4±6.5%.

% DFI values in a range of 27-30% have been reported to be the point at which men are infertile [Evenson and Wixon, Theriogenology 2006; 65:979-991]. It is therefore significant that the average % DFI value in the Cy+AS101 group was below this range.

Results of Evaluation of Male Fertility:

As shown in FIG. 10, administration of Cy resulted in a significant reduction in the percentage of impregnated females, from 93.3±6.1% in the control group to 20.0±4.2%, whereas the percentage of impregnated females in the Cy+AS101 group dropped only slightly to 80.0±8.2%.

As shown in FIG. 11, the litter size of females which did achieve pregnancy was reduced by Cy, from 9.9±1.0 in the control group to 4.3±1.4, whereas litter size was reduced only slightly by Cy+AS101 to 8.3±0.6.

Effect of Treatments on Akt and GSK-3β Phosphorylation:

Phosphorylation of Akt and GSK-3β was determined using gel electrophoresis and Western blotting, as described in the Materials and Methods section.

Akt and GSK-3β phosphorylation has been shown to be partly responsible for AS101-induced protection of neuronal cells [Kalechman et al., J Am Soc Nephrol 2003; 14:620-630; Okun et al., J Neurochem 2007; 102:1232-1241; Sredni et al., FASEB J 2007; 21:1870-1883]. Activation of the Akt/GSK-3β pathway is known to play a crucial role in DNA repair [Kao et al., J Biol Chem 2007; 282:21206-21212].

Previous studies have shown that Akt activation can induce radio- and chemo-protection by enhancing spermatogenic stem cell survival and by increasing stem cell self-renewal [Rasoulpour et al., Endocrinology 2006; 147:4213-4221]. GSK-3β regulates cell metabolism, cell cycle and cell fate through the phosphorylation of a diverse array of substrates. Akt inhibits GSK-313 activity by phosphorylation at Ser9. Guo et al. [J Androl 2003; 24:332-342] have shown evidence suggesting that GSK-3β has a critical role in mammalian meiosis and spermatogenesis, and its inhibition results in down-regulation of meiotic DNA synthesis.

As shown in FIGS. 12A and 12B, AS101 induced a significant increase in the average level of phosphorylated Akt (pAkt) to 175.3±28.4% relative to the PBS group (P<0.05), Cy decreased the level of pAkt to 55.3±25.6% of the PBS group (P<0.05), and Cy+AS101 resulted in an increase in the level of pAkt to 164.3±5.7% of the PBS group, which was significantly higher (P<0.05) than in both the PBS and Cy groups.

As shown in FIGS. 13A and 13B, AS101 also caused a significant increase in the level of phosphorylated GSK-3β (pGSK-3β) to 130.3±16.9% of the PBS group (P<0.05), Cy decreased the level of pGSK-313 to 36.4±12.5% (P<0.05), and Cy+AS101 resulted in an increase in the level of pGSK-30 to 114.5±9.2% of the PBS group, which was significantly higher (P<0.05) than in both the PBS and Cy groups.

The above results indicate that AS101 is effective in protecting against cyclophosphamide-induced testicular damage, DNA damage and infertility.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1-39. (canceled)

40. A method of maintaining and/or augmenting fertility in a male subject undergoing chemotherapy and/or radiotherapy, the method comprising:
(a) administering to said male subject a therapeutically effective amount of a chemotherapeutic agent and/or radiation;
(b) administering to said male subject a gonadal-protective amount of a tellurium-containing compound;
(c) instructing said male to refrain from conceptive sex for a predetermined time period following administration of said chemotherapeutic agent and/or radiation.

41. A tellurium-containing compound identified for use in maintaining and/or augmenting fertility in a male subject undergoing chemotherapy and/or radiotherapy, the tellurium-containing compound being for use in combination with a chemotherapeutic agent and/or radiation such that said male subject receiving said chemotherapeutic agent and/or radiation and the tellurium-containing compound is instructed to refrain from conceptive sex for a predetermined time period following administration of said chemotherapeutic agent and/or radiation.

42. The tellurium-containing compound of claim 41, further comprising a pharmaceutically acceptable carrier.

43. The tellurium compound of claim 41, being such that after the end of said predetermined time period, said male subject can practice reproductive activity with a female partner.

44. The tellurium-containing compound of claim 43, wherein said reproductive activity is selected from the group consisting of conceptive sex and assisted reproduction.

45. The method of claim 40, wherein said pre-determined time period is selected from the group consisting of less than 4 months, less than 100 days, less than 3 months, less than 2 months, less than 1 month and less than 6 months.

46. The method of claim 40, wherein said predetermined time period is such that values of a sperm count, functionality and/or appearance of said male subject at the end of said time period are at least close to normal or reference values.

47. The method of claim 46, wherein said predetermined time period is such that a sperm DNA structure of said male subject at the end of said time period is at least close to normal.

48. The method of claim 46, further comprising, prior to administering to said male subject said chemotherapeutic agent and/or radiation:
determining values of a sperm count, functionality and/or appearance of said male subject, said values being said reference values.

49. The method of claim 48, further comprising, subsequent to instructing said male subject to refrain from conceptive sex:
determining values of a sperm count, functionality and/or appearance of said male subject; and
determining if said values of a sperm count, functionality and/or appearance of said male subject are at least close to said reference values.

50. The tellurium-containing compound of claim 41, wherein said tellurium-containing compound has a general formula selected from the group consisting of: 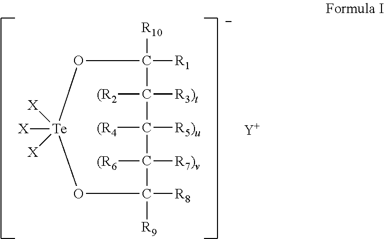
a compound having general Formula 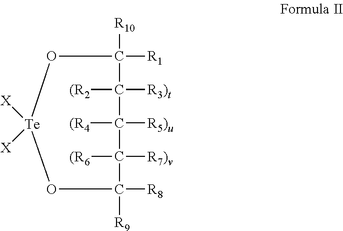
a compound having general Formula 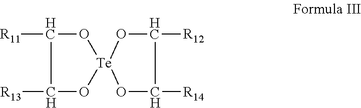
and
a compound having general Formula IV: 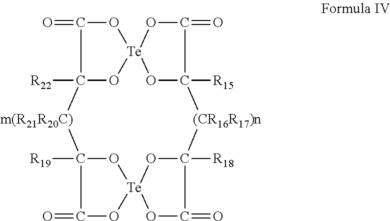
wherein:
each of t, u and v is independently 0 or 1;
each of m and n is independently 0, 1, 2 or 3;
Y is selected from the group consisting of ammonium, phosphonium, potassium, sodium and lithium;
X is a halogen atom; and
each of R₁-R₂₂ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfonamido.

51. The tellurium-containing compound of claim 50, wherein said tellurium-containing compound has said general Formula I.

52. The tellurium-containing compound of claim 51, wherein t, u and v are each 0.

53. The methodtellurium-containing compound of claim 52, wherein each of R₁, R₈, R₉ and R₁₀ is hydrogen.

54. The tellurium-containing compound of claim 50, wherein X is chloro.

55. The method, use, pharmaceutical composition or tellurium-containing compound of claim 54, wherein Y is ammonium.

56. The tellurium-containing compound of claim 50, wherein said tellurium-containing compound is ammonium trichloro(dioxyethylene-O,O′)tellurate (AS101).

57. The tellurium-containing compound of claim 50, wherein said compound has said general Formula IV.

58. The method of claim 40, wherein the tellurium-containing compound is at least one of ammonium trichloro(dioxyethylene-O,O′)tellurate (AS101) and SAS.

59. The tellurium-containing compound of claim 41, wherein said tellurium-containing compound is SAS.
